(19) United States
(12) Patent Application Publication
(10) Pub. No.: US 2012/0310155 A1
(43) Pub. Date: Dec. 6, 2012

(54) APPARATUS AND METHOD FOR DERMAL DELIVERY

(76) Inventors: Jeremy Heiser, Salt Lake City, UT (US); John Howard Gordon, Salt Lake City, UT (US)

(21) Appl. No.: 13/485,638

(22) Filed: May 31, 2012

Related U.S. Application Data
(60) Provisional application No. 61/491,637, filed on May 31, 2011.

(51) Int. Cl. A61M 5/31; A61M 5/00

(52) U.S. Cl. 604/82; 604/272; 604/173; 604/180; 604/187; 604/151; 604/506

(57) ABSTRACT

A dermal delivery apparatus includes a pad having a surface to interface with an outer layer of skin. The dermal delivery apparatus also includes a micro structure coupled to the pad and configured to penetrate and deliver an agent beneath the outer layer of skin. The dermal delivery device also includes a base at least partially surrounding the micro structure. The base is coupled to the pad and protrudes away from the surface of the pad to apply surface tension to the skin.

### CROSS-REFERENCE TO RELATED APPLICATIONS

This application claims the benefit of priority of U.S. Provisional Patent Application 61/491,637 entitled, “Dermal Delivery System” filed on May 31, 2011. The contents of this application are incorporated by reference herein in their entirety.

### BACKGROUND

Injection of substances through the dermal layer is a standard industry practice in the medical field. In some instances the injection is a momentary syringe injection while, in other instances, a more sustained, relatively long-term injection is more appropriate. In many conventional injections, the hollow needle is inserted deep enough into the patient to reach nerves and cause pain. Additionally, many conventional systems do not have a way of controlling the depth of the needle at the injection site. Such systems are sensitive to pressure and stresses that accompany movement of the site and movement around the injection equipment. This can lead to a level of discomfort in the patient, less efficient administration of medicines, and longer healing times due to irritation and site damage from lack of depth and motion control.

Compatibility of systems is also a valuable advantage in any field. In many conventional delivery systems, components must be matched with other components from a specific manufacturer and type for the system to function properly. It is desirable to have a system function properly with a wider range of components to increase flexibility and utility.

### SUMMARY

According to described embodiments, a dermal delivery apparatus is disclosed. The dermal delivery apparatus includes a pad having a surface to interface with an outer layer of skin. The dermal delivery apparatus also includes a micro structure coupled to the pad and configured to penetrate and deliver an agent beneath the outer layer of skin. The dermal delivery device also includes a base at least partially surrounding the micro structure. The base is coupled to the pad and protrudes away from the surface of the pad to apply surface tension to the skin.

Embodiments for a method for delivering an agent beneath an outer layer of skin are also described. The method includes applying a dermal delivery device to the outer layer of the skin creating tension in the skin to control a penetration depth beneath the outer layer of the skin, penetrating the outer layer of the skin with a micro structure, and delivering the agent beneath the outer layer of the skin through the micro structure. Other embodiments of the method are also described herein.

Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

FIG. 1 depicts a schematic diagram of one embodiment of a dermal delivery apparatus with a tube inlet.

FIG. 2 depicts a schematic diagram of one embodiment of a dermal delivery apparatus with adhesive surfaces and a luer inlet receiver.

FIG. 3 depicts a schematic diagram of another embodiment of a dermal delivery apparatus with an adhesive overlay and a plural inlet receiver.

FIG. 4 depicts a schematic diagram of one embodiment of a fill adapter coupled to a dermal delivery apparatus.

FIG. 5 depicts another schematic diagram of the fill adapter of FIG. 4.

FIG. 6 depicts a flow chart diagram of one embodiment of a method for delivering an agent beneath an outer layer of skin.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

Embodiments of the present invention have been developed in response to the present state of the art and, in particular, in response to the problems and needs in the art that have not yet been fully solved by currently available structures and methods. Accordingly, embodiments of the invention have been developed to provide structures and methods to overcome various shortcomings of the prior art. The features and advantages of various embodiments of the invention will become more fully apparent from the following description and appended claims, or may be learned by practice of the invention as set forth hereinafter.

While many embodiments are described herein, at least some embodiments relate to a dermal delivery apparatus. Embodiments of the dermal delivery apparatus may be stand-alone units or may be configured to interface with existing delivery systems. The dermal delivery apparatus can deliver an agent below the skin of a patient without impacting nerves or causing significant pain. The dermal delivery apparatus can deliver a relatively short dose similar to a conventional shot or a relatively long or complex dosing similar to a conventional intervenous delivery system.

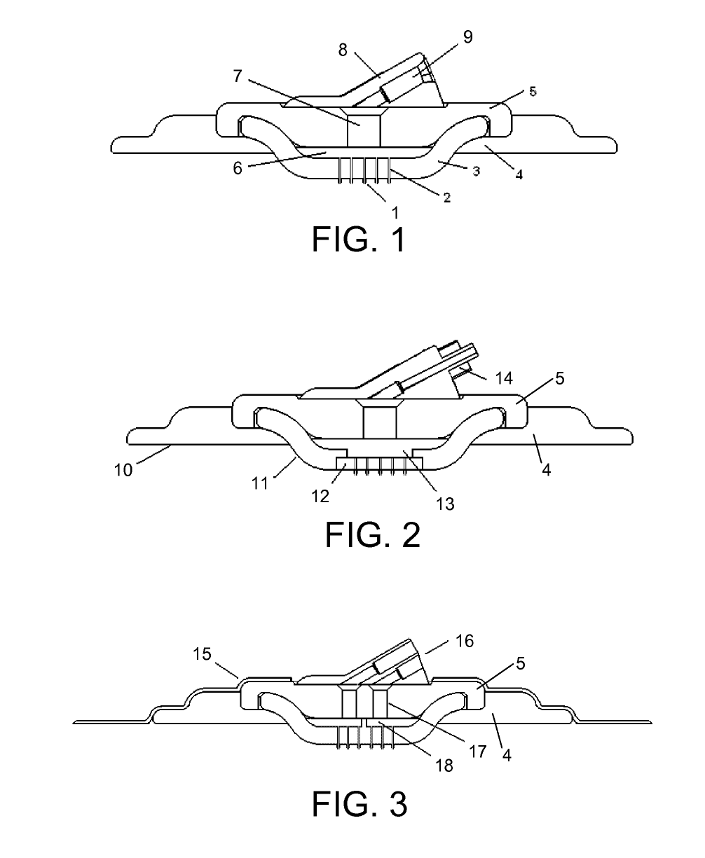

FIG. 1 depicts a schematic diagram of one embodiment of a dermal delivery apparatus with a tube inlet 9. The illustrated embodiment includes at least one penetrating micro structure 1, at least one micro structure through-hole 2, a base 3, a pad 4, a channeled cap 5, a distribution cavity 6, a channel 7, an inlet receiver 8, and a tube inlet 9.

In the illustrated embodiment, an array of penetrating micro structures 1 is shown. In some embodiments, only one penetrating micro structure 1 is used. In other embodiments, a plurality of micro structures 1 may be incorporated. A plurality of penetrating micro structures 1 may be configured to reach a single uniform depth while some embodiments may be configured to reach two or more levels of penetration. The penetrating micro structures 1 may have a variety of geometries. Some of these geometries may include hollow micro needles, solid micro needles, micro blades, or micro abraders. In some embodiments the plurality of micro structures 1 are all of a single geometry. In other embodiments, the penetrating micro structures 1 are of differing geometries. In some embodiments, at least one penetrating micro structure 1, has a geometry designed to divert tissue away from the aperture of the micro structure 1 during penetration and administration of an agent. In some embodiments, the micro structure 1 maximizes the amount of tissue that is directly exposed to the substance delivered through the penetrating micro structure 1. In some embodiments, the penetrating micro structure 1 is aligned perpendicular to the plane of the skin. In other embodiments, the plurality of penetrating micro structures 1 may be aligned at one or more angles to produce an effect on the penetration of the skin or on the delivery or dispersion of the agent beneath the skin.

The benefit of the micro structure 1 is that it can penetrate the outer layer of skin while not going so deep that nerves are reached. For example, the micro structure 1 may penetrate between approximately 100 to 1,500 microns below the outer layer of the skin. Thus the biological barrier of the stratum corneum is bypassed, the capillaries of the papillary dermis are accessed, the nerves may not be irritated (so the patient may not suffer pain), and the integrity of the skin is minimally disturbed by the penetration and delivery process. The additional benefits of using at least one penetrating micro structure 1 are that the features of the micro structure 1 serve to divert tissue away from the aperture during penetration, hold the tissue a distance from the aperture throughout the delivery process, control the depth of delivery into the skin and allow for an increase in the amount of tissue that is directly exposed to the substance delivered. These benefits allow for a more efficient and effective delivery of the agent and, in turn, systematic absorption.

The lumen or internal path within the penetrating micro structure 1 allows an agent to be delivered below the outer layer of skin. In some embodiments, the lumen measures in the range of approximately 5 to 1,000 microns in diameter. The lumen may be centered within the micro structure 1 or positioned off-center. The lumen may be parallel to the axis of the micro structure 1 or at an angle relative to the axis of the micro structure 1.

Also shown in the illustrated embodiment is the micro structure to through-hole 2. The through-hole 2 allows the micro structure 1 to pass though the base 3. In some embodiments, the micro structure 1 is formed as a unified structure with the base 3. In other embodiments, the micro structure 1 is a separate element which is secured in the through-hole 2 within the base 3 by molding, adhesive, or other means of securing. In some embodiments, the through-hole 2 size ranges from 5 to 4,000 microns in diameter. In some embodiments, the through-hole 2 may be tapered, have a varied diameter, or have a circular or non-circular diameter. In some embodiments, the through-hole 2 allows the user to fill the apparatus by drawing an agent in through the through-hole 2. In some embodiments, the through-hole 2 has a directional valve to control flow of the agent. Other embodiments include more or less functionality with the through-hole 2.

The illustrated embodiment of FIG. 1 also depicts the base 3. The base 3 may be made of rigid or semi-rigid material suitable for contact with skin. The base 3 has a protruded geometry in that it protrudes away from pad 4. In some embodiments, the protruded geometry of the base 3 forms a dome. In other embodiments, the geometry of the base 3 is a square. Other embodiments may include a circular, triangular, or other geometric shape. The base 3 may have a generally planar, concave, or convex shape. The base 3 applies tension to the underlying skin at the location of the micro structure 1. The applied tension provides for a more uniform penetration of the skin by the micro structure 1. Additionally, the geometry of the base 3 focuses and sustains the force of the micro structure 1 to ensure that penetration is singular and sustained without disengaging and re-engaging to causes multiple punctures due to movement or external forces.

The pad 4 reduces motion relative to the underlying skin. The reduction in relative motion helps to reduce shear forces on the penetrating micro structure 1 and maintains the micro structure 1 in place at the desired depth. Maintaining the position of the penetrating micro structure 1 prevents irritation of the delivery site due to multiple punctures from movement of the micro structure 1. The pad 4 may also provide a conforming interface. In some embodiments, the base 3 and cap 5 are rigid to provide structural support and a place to attach agent to transfer mechanisms. The pad 4 helps conform the more rigid structures of the dermal delivery apparatus to the skin surface. In this manner, the pad 4 protects the skin from the edges of the cap 5. The pad 4 also serves to make the dermal delivery apparatus adjustable relative to the skin and allows for greater control of the depression of the base 3 into the skin. For example, the pad 4 thickness and pliability may be varied in order to accommodate the tautness or other features of the skin in order to maximize the effectiveness of the connection between the dermal delivery apparatus and the skin.

In some embodiments, the pad 4 is a relatively flexible material. In some embodiments, the pad 4 is compatible with an adhesive. For some applications it may be advantageous to have a tapered geometry so that tapes or wraps may be placed over the pad 4 to prevent motion relative to the skin of the patient. The tapered geometry may reduce discomfort from pressure at the edges of the pad 4 when wrapped or otherwise secured. The illustrated embodiment shows a step-down type taper. Other embodiments may use a curved, flat, or other style of taper or edge treatment. In some embodiments, the pad 4 may be made of two layers; a relatively rigid moldable frame layer and a flexible cushion layer. A multi-layer embodiment may enhance the security of the apparatus by allowing a more rigid frame to be fit to the contours of the area to receive the injection while the softer portion may provide comfort and additional resistance to shearing forces at the micro structure 1 during use. Other embodiments may include other variations of the pad 4.

The illustrated embodiment also includes the channeled cap 5. The channeled cap 5 includes an internal channel 7 which directs an agent to the micro structure 1. In some embodiments, the channeled cap 5 couples to the base 3 to form a seal to prevent the substance from leaking out between the base 3 and the channeled cap 5. In some embodiments, the channeled cap 5 is made of a rigid material. In other embodiments, the channeled cap 5 is made semi-rigid material.

The distribution cavity 6 is formed between the channeled cap 5 and the base 3. In some embodiments, the distribution cavity 6 allows for the agent coming from the channeled cap 5 to enter the penetrating micro structure 1. In some embodiments, the distribution cavity 6 distributes the fluid pressure evenly over multiple penetrating micro structures 1. The distribution of pressure allows for more even and effective distribution at the delivery site. In some embodiments, the distribution cavity 6 has a structural feature or fluid dynamic effect to facilitate mixture of the agent as it passes through the distribution cavity 6. In one embodiment, the cap 5 interfaces directly with the base 3, thereby eliminating the need for the distribution cavity 6.

The channel 7 is formed within the channeled cap 5. In some embodiments, the channel 7 has a circular cross-section. In other embodiments, the channel 7 has a square, triangular, or other geometric or irregular cross-section. In some embodiments, the channel 7 has a uniform geometry throughout. In other embodiments, the cross-section of the channel 7 may vary along its length. For example, in some embodiments, the channel 7 may have a tapered or reverse-tapered cross-section. Some embodiments of the channel 7 my produce a fluid dynamic effect on the agent passing through it. For example, the channel 7 geometry may produce a turbulent mixing effect on the substance as it passes into the distribution cavity 6. In other embodiments, the channel 7 may produce other effects.

The inlet receiver 8 of FIG. 1 attaches to the channeled cap 5. In some embodiments, the inlet receiver 8 and the channeled 5 form a seal to prevent leakage. Several embodiments of the inlet receiver 8 will be described with reference to the following figure descriptions. In the illustrated embodiment of FIG. 1, the inlet receiver 8 facilitates coupling an agent transfer device to the inlet receiver 8 to direct an injection substance into the apparatus. In different embodiments, the agent transfer device may take several different forms. For example, the agent transfer device may be a pump, a syringe, or the illustrated embodiment, a medical tube. In the illustrated embodiment of FIG. 1, the tube inlet 9 is formed within the inlet receiver 8 to allow fluid communication between the agent transfer device and the channeled cap 8. In some embodiments, the tube inlet 9 has a tapered geometry to secure a tube inserted into the tube inlet 9. In some embodiments the tube inlet 9 has a flared opening to facilitate ease of tube insertion. Some embodiments of the tube inlet 9 may have a surface treatment or feature within the tube inlet 9 to facilitate forming a seal with an inserted tube.

FIG. 2 depicts a schematic diagram of one embodiment of a dermal delivery apparatus with adhesive surfaces 10 and 11 and a luer inlet receiver 14. The illustrate embodiment of FIG. 2 includes pad adhesive 10, a base adhesive 11, a substrate 12, a base channel 13, and a luer inlet receiver 14. In the illustrated embodiment, the pad adhesive 10 is applied to the skin-side of the pad 4 of FIG. 1. The pad adhesive 10 further facilitates reduction of motion relative to the skin. In some embodiments, the pad adhesive 10 also prevents relative shearing movement of the pad 14 with respect to the skin of the patient which reduces stress on the penetrating micro structure 1, which, in turn, reduces the irritation of the injection site of the patient. In some embodiments, the pad adhesive 10 is an adhesive compound applied directly to the pad 4. In other embodiments, the pad adhesive 10 is a separate film with an adhesive compound applied to both sides of the film with one side adhered to the pad 4. In some embodiments, the pad adhesive 10 is suitable for repeated or long-term applications on a patient's skin.

The illustrated embodiment also includes the base adhesive 11. Many embodiments of the base adhesive 11 are similar to the above-described embodiments of the pad adhesive 10. In some embodiments, the base adhesive 11 allows the skin to slide relative to the base 3 before setting and securing the skin in place. This allows the skin to be tensioned uniformly and without significant skin discomfort from the tension created by the base 3.

The substrate 12 included in the illustrated embodiment of FIG. 2 secures the penetrating micro structure 1. In some embodiments, the substrate 12 containing the penetrating micro structure 1 is permanently fixed to the base 3. In other embodiments, the substrate 12 is modular and may be removed for cleaning or replacement. In some embodiments, the substrate 12 covers the micro structures 1 to provide protection before use but deforms to expose the micro structures 1 upon application to a delivery site on a patient. In other embodiments, the substrate 12 is rigid to prevent deflection or misalignment of the micro structures 1.

In the illustrated embodiment, the base channel 13 forms a portion of the delivery path for the agent to be injected. The channel 13 forms a path through at least a portion of the base 3. In some embodiments, the channel 13 distributes the agent to the penetrating micro structure 1. In the illustrated embodiment, the channel 13 secures the substrate 12 containing the penetrating micro structure 1 within the base 3.

The luer inlet receiver 14 interfaces with a source of the agent to be injected with a luer type seal. The luer inlet receiver 14 may be the male or female portion of the luer fitting. The luer inlet receiver 14 may be particularly effective in allowing the apparatus to be coupled to a conventional system such as a syringe. For example, a conventional syringe may be attached to the luer inlet receiver 14 to replace the conventional intramuscular needle. The compatibility of the luer fitting of the luer inlet receiver 14 would allow an agent to be loaded into a syringe but delivered without the pain or discomfort of a conventional intramuscular needle. In the illustrated embodiment, the luer inlet receiver 14 is oriented at an angle. In other embodiments, the luer inlet receiver 14 may be oriented perpendicular or parallel to the skin. Other embodiments may incorporate other luer inlet receiver 14 angles to facilitate specific applications.

FIG. 3 depicts a schematic diagram of another embodiment of a dermal delivery apparatus with an adhesive overlay 15 and a plural inlet receiver 16. The illustrated embodiment of FIG. 3 includes an adhesive overlay 15, a plural inlet receiver 16, plural channels 17, and a partitioned distribution cavity 18.

In the illustrated embodiment, the adhesive overlay 15 is applied over the pad 4 and the channeled cap 5. In other embodiments, the adhesive overlay 15 may cover more or less of the apparatus. In one embodiment, the adhesive overlay 15 may be used exclusive of other adhesive applied to the pad 4 or the base 3. In other embodiments, the adhesive overlay 15 may be used in conjunction with other adhesives. The adhesive overlay 15 is applied to the patient's skin during use.

In the illustrated embodiment, the plural inlet receiver 16 forms a portion of the substance delivery path. The plural inlet receiver 16 allows for more than one source to supply agents or agent components for delivery. In some embodiments, the plural inlet receiver 16 facilitates multiple substance types for mixed delivery. For example, the substances could be mixed directly before injection, injected separately, injected individually and at separate times, injected at different flow rates. The plural inlet receiver 16 also allows for independent or simultaneous staged delivery of one or more substances.

The plural channels 17 are similar to the channel 7 in the channel cap 5 of FIG. 1 except that the plural channels 17 allow for multiple substance flow paths. The functionality and advantage of multiple flow paths is described above with respect to the plural inlet receiver 16.

The partitioned distribution cavity 18, used in conjunction with the plural inlet receiver 16, facilitates the functionality described above with respect to the plural inlet receiver 16. Further, in some embodiments, the partitioned distribution cavity 18 may allow for additional functionality. For example, the partitioned distribution cavity 18 facilitates partial mixing, selection of portions of the penetrating micro structures for specific substance delivery, and flow rate control from the partitioned distribution cavity 18. Other embodiments of the partitioned distribution cavity 18 may incorporate more or less functionality.

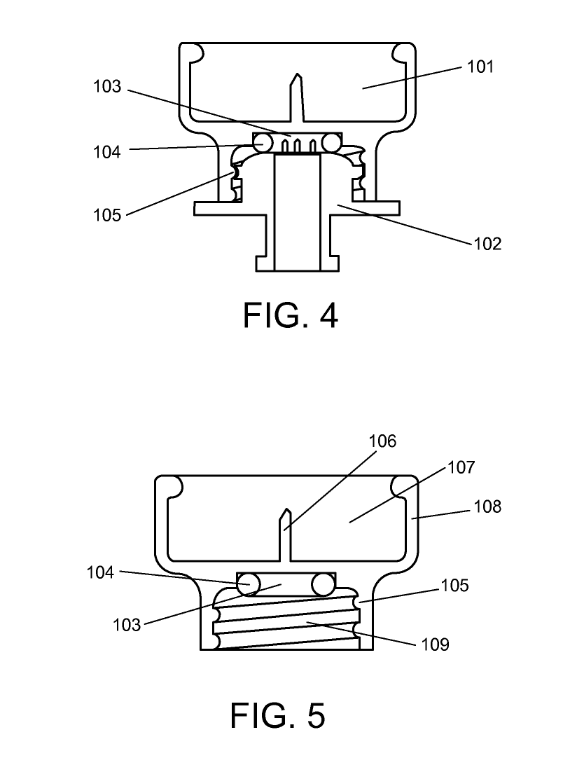

FIG. 4 depicts a schematic diagram of one embodiment of a fill adaptor 101 coupled to a dermal delivery apparatus 102. The illustrated embodiment includes a vial adaptor 101, a dermal delivery apparatus 102, a fluid path 103, a seal 104, and an integration structure 105. The vial adaptor 101 couples to the apparatus 102 to facilitate transfer of an injection fluid from a conventional vial to the apparatus 102.

The fluid path 103 is formed by coupling the adaptor 101 to the apparatus 102. In some embodiments, the fluid path 103 allows for mixture of the agent. In other embodiments, the fluid path 103 facilitates mixing a solid agent component with a fluid agent component. For example, the apparatus 102 may be prefilled with a fluid agent component prior to coupling the apparatus 102 to the adaptor 101. The adaptor 101 is coupled to the apparatus 102 and the fluid agent component is injected through the adaptor 101 and into a vial carrying the solid agent component (likely in powder form). The mingled agent components are drawn back through the adaptor 101 and at least partially mix within the fluid path 103 before refilling into the apparatus 102 to be later injected into a patient.

The seal 104 forms a temporary seal between the adaptor 101 and the apparatus 102 to form the fluid path 103. In some embodiments, the seal 104 is permanently affixed to the adaptor 101. In other embodiments, the seal 104 is floating and easily removed to facilitate replacement or cleaning/sterilization. The seal 104 may take several forms. For example, the seal 104 may be an o-ring, gasket, surface coating, opposing surfaces, or combination of different types of seals.

The integration structure 105 facilitates temporary integration of the adaptor 101 with a complementary integration structure of the apparatus 102. In one embodiment, the integration structure 105 contacts a complementary integration structure located on the base 3. The integration structure 105 provides compression to maintain the closure provided by the seal 104. The integration structure 105 may take several forms. For example, the integration structure 105 may be a threaded interface, notch-and-notch receptacle, snap feature, interlocking notch and groove, friction fit, interference fit, bump and depression, temporary adhesive, latch feature, or combination of multiple integration structures. Additionally, some embodiments of the integration structure 105 may provide additional sealing to prevent leakage.

FIG. 5 depicts another schematic diagram of the fill adaptor 101 of FIG. 4. The illustrated embodiment of FIG. 5 includes an access spike 106, a vial receiver portion 107, a vial guide 108, and a dermal delivery apparatus receiver portion 109. The access spike 106 accesses a conventional medical vial by puncturing the vial septum and providing a fluid path via fluid channel to remove the fluid from the vial. In one embodiment, the access spike 106 has one or more lumens placing the aperture of the spike 106 in communication with the fluid path 103. The access spike 106 may have several different geometries. For example, the access spike 106 may be conical, tapered, beveled needle tip, angled needle tip, multi-angled faced, pointed, blunt, or have a combination of the aforementioned or other geometries. In the illustrated embodiment, the access spike 106 has a channel running the length of the access spike 106 to facilitate removal of substantially all of the agent material from the vial. In another embodiment, the access spike 106 is a hollow spike with an opening situated to facilitate removal of the agent from the vial. The opening or aperture of the access spike 106 may be on the tip of the spike 106 or at another location along the access spike 106 to extract fluid from a vial through the vial septum.

The vial receiver portion 107 receives and supports the accessible end of a vial. The vial guide 108 aligns the vial to allow the spike 106 to puncture the vial and extract the fluid. The vial guide 108 may be made of rigid or semi-rigid material. In some embodiments, the vial guide 108 may be shorter than the to access spike 106. In other embodiments, the vial guide 108 is equal to or longer than the access spike 106. In some embodiments, the vial guide 108 is uniform along its depth. Other embodiments of the vial guide 108 are staged or have varied diameters along its depth. In some embodiments, the vial guide 108 has bumps, rings, or notches to further facilitate insertion and removal of a vial. In some embodiments, the vial guide 108 is a permanent and unified portion of the vial adaptor 101. In other embodiments, the vial guide 108 is temporarily affixed to the adaptor 101. In the illustrated embodiment, the vial guide 108 includes a ring at its outer edge to close around and secure the vial during extraction. This ring may be deformable or rigid.

The dermal delivery apparatus receiver portion 109 receives and secures the dermal delivery apparatus 102 through use of the integration structure 105 as described above.

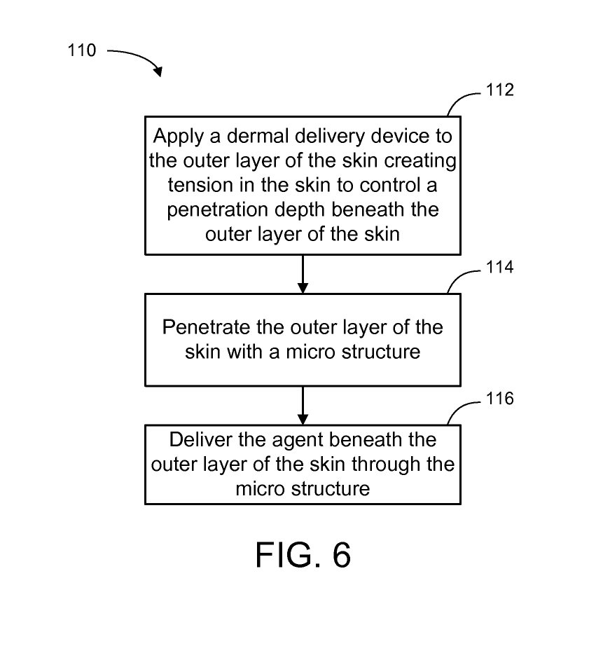

FIG. 6 depicts a flow chart diagram of one embodiment of a method 110 for delivering an agent beneath an outer layer of skin. The illustrated method 110 includes applying 112 a dermal delivery apparatus to the outer layer of the skin creating tension in the skin to control a penetration depth beneath the outer layer of the skin. The method 110 also includes penetrating 114 the outer layer of the skin with a micro structure. The method 110 also includes delivering 116 the agent beneath the outer layer of the skin through the micro structure. Other embodiments of the method 110 may include fewer or more operations.

In one embodiment, the forgoing steps are preceded by the dermal delivery apparatus being attached to a substance or agent source such as a vial or other source. As the vial receiver portion is attached to a vial, the access spike penetrates a membrane of the vial allow the agent to enter an agent transfer device such as a syringe. In another embodiment, the inlet receiver of the dermal delivery apparatus may be a luer which is attached to an agent source such as a syringe. The syringe then pushes the agent into an agent transfer device such as a pump for later delivery to the skin. The adaptor, may then be removed from the dermal delivery apparatus.

While many embodiments are described herein, at least some embodiments present a technical application with certain advantages over conventional technologies. As one example, the disclosed dermal delivery apparatus requires between one half and one fifth of the volume required for a conventional intramuscular delivery system to achieve the same effect. Additionally, the advantage of using the skin's immune response system is of great benefit to mass populations in the event of a pandemic outbreak or post-disaster situation. Human skin contains a dense population of first-line immune cells (dendritic cells) and is thus ideal for delivery of an injected agent.

Other potential advantages of the disclosed embodiments are ease and safety of use. The disclosed system requires minimal training for a standardized delivery procedure. This, in turn, allows vaccine and drug manufacturers to implement dermal delivery dosing guidelines for commercially available vaccines and drugs; thereby, allowing for the user of dose-sparing strategies. This would reduce agent shortage issues and would improve control of distribution and administration.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner. Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A dermal delivery apparatus comprising:
a pad having a surface to interface with an outer layer of skin;
a micro structure coupled to the pad and configured to penetrate and deliver an agent beneath the outer layer of skin; and
a base at least partially surrounding the micro structure, wherein the base is coupled to the pad and protrudes away from the surface of the pad to apply surface tension to the skin.

2. The dermal delivery apparatus of claim 1, wherein the base comprises a dome.

3. The dermal delivery apparatus of claim 1, wherein the base comprises a ring.

4. The dermal delivery apparatus of claim 1, wherein the base comprises a cylinder.

5. The dermal delivery apparatus of claim 1, wherein the base comprises a square.

6. The dermal delivery apparatus of claim 1, wherein the base comprises a pyramid.

7. The dermal delivery apparatus of claim 1, wherein the base comprises a triangle.

8. The dermal delivery apparatus of claim 1, wherein the base is configured to apply the surface tension to the skin to facilitate a uniform penetration depth of a plurality of micro structures.

9. The dermal delivery apparatus of claim 1, further comprising a channeled cap coupled to the base, the channeled cap comprising a channel to direct the agent to the micro structure.

10. The dermal delivery apparatus of claim 9, wherein the channeled cap is configured to substantially mix the agent with another substance within the dermal delivery apparatus.

11. The dermal delivery apparatus of claim 9, wherein the channeled cap is configured to prevent mixture of a multi-part agent within the dermal delivery apparatus.

12. The dermal delivery apparatus of claim 9, wherein the pad is configured to protect the skin from stress concentration and shear force caused by forces applied to the dermal delivery apparatus.

13. The dermal delivery apparatus of claim 9, wherein the pad comprises an adhesive to secure the dermal delivery apparatus relative to the skin.

14. The dermal delivery apparatus of claim 9, further comprising an inlet receiver coupled to the channeled cap opposite the base, the inlet receiver to facilitate connection of an agent transfer device.

15. The dermal delivery apparatus of claim 14, wherein the agent transfer device comprises a syringe.

16. The dermal delivery apparatus of claim 14, wherein the agent transfer device comprises a pump.

17. The dermal delivery apparatus of claim 14, wherein the agent transfer device comprises medical tubing.

18. The dermal delivery apparatus of claim 14, wherein the agent transfer device comprises an agent generator, wherein the agent generator generates the agent at substantially the dermal delivery apparatus.

19. The dermal delivery apparatus of claim 1, further comprising a fill adapter to couple to the base, the fill adapter comprising a seal interface to form at least a partial seal to facilitate transfer of an agent into the dermal delivery apparatus through the micro structure, wherein the fill adapter is configured to connect the dermal delivery apparatus to an agent supply to fill the dermal delivery apparatus.

20. The dermal delivery apparatus of claim 19, wherein the fill adapter further comprises an access spike to pass through an access point of the agent supply and transport the agent from the agent supply to the dermal delivery apparatus.

21. A method for delivering an agent beneath an outer layer of skin, the method comprising:
applying a dermal delivery device to the outer layer of the skin creating tension in the skin to control a penetration depth beneath the outer layer of the skin;
penetrating the outer layer of the skin with a micro structure; and
delivering the agent beneath the outer layer of the skin through the micro structure.

22. The method of claim 21, further comprising filling a delivery apparatus, wherein filling the delivery apparatus comprises attaching a fill adapter to the delivery apparatus, wherein the fill adapter comprises a seal to contact the delivery apparatus and an access spike to be compatible with a conventional agent vial, and drawing the agent from the conventional agent vial and into the delivery apparatus through the fill adapter.

23. The method of claim 21, wherein creating tension in the skin comprises contacting a base having a protruded geometry to the skin to displace the skin resulting in the tension in the skin.

24. The method of claim 23, wherein delivering the agent comprises directing the agent through a channeled cap configured to direct the agent through a channel of the channeled cap to the micro structure.

25. The method of claim 24, wherein the channeled cap is configured to substantially mix the agent with another substance within the dermal delivery device.

26. The method of claim 25, wherein the channeled cap is configured to separate a first portion of the agent from a second portion of the agent.

27. The method of claim 21, further comprising at least partially protecting the skin from stress concentration and shear force due to pressure and motion with a pad.

28. The method of claim 27, wherein at least a portion of the protection comprises an adhesive to substantially prevent movement relative to the skin.

29. The method of claim 25, further comprising an inlet receiver to direct the agent to the channeled cap and to facilitate connection of a substance transfer device.

30. The method of claim 29, wherein the substance transfer device comprises a syringe.

31. The method of claim 29, wherein the substance transfer device comprises a pump.

32. The method of claim 29, wherein the substance transfer device comprises medical tubing.

33. The method of claim 29, wherein the substance transfer device comprises an agent generator to generate the agent within the dermal delivery device.

34. A dermal delivery apparatus comprising:
a pad having a surface to interface with an outer layer of skin, wherein the pad is configured to protect the skin from stress concentration and shear force caused by forces applied to the dermal delivery apparatus;
a micro structure coupled to the pad and configured to penetrate and deliver an agent beneath the outer layer of skin;
a base at least partially surrounding the micro structure, wherein the base is coupled to the pad and protrudes away from the surface of the pad to apply surface tension to the skin to facilitate a uniform penetration depth of a plurality of to micro structures; and
a channeled cap coupled to the base, the channeled cap comprising a channel to direct the agent to the micro structure.

35. A dermal delivery apparatus comprising:
a micro structure configured to penetrate and deliver an agent beneath an outer layer of skin;
a base at least partially surrounding the micro structure, wherein the base comprises a protruded geometry configured to apply surface tension to the skin and an integration structure; and
a fill adapter coupled to the integration structure of the base, wherein the fill adapter is configured to facilitate connection of the dermal delivery apparatus to an agent vial and transportation of the agent from the agent vial into the dermal delivery apparatus.

36. The dermal delivery apparatus of claim 35, wherein the fill adapter comprises a seal interface to form at least a partial seal with the base to transfer the agent into the dermal delivery apparatus through the micro structure.

37. The dermal delivery apparatus of claim 35, wherein the fill adapter comprises a collar configured to secure an agent vial

38. The dermal delivery apparatus of claim 35, wherein the fill adapter comprises an access spike to access the agent vial at an access point of the vial and transport the agent from the vial to the dermal delivery apparatus.

39. The dermal delivery apparatus of claim 35, wherein the integration structure comprises a luer fitting.

40. The dermal delivery apparatus of claim 35, wherein the integration structure comprises a snap feature.

41. The dermal delivery apparatus of claim 35, wherein the integration structure comprises a notch-and-notch fitting.

42. The dermal delivery apparatus of claim 35, wherein the integration structure comprises an interlocking notch-and-groove fitting.

43. The dermal delivery apparatus of claim 35, wherein the integration structure comprises a temporary adhesive.

44. The dermal delivery apparatus of claim 35, wherein the integration structure comprises a friction fitting.

45. The dermal delivery apparatus of claim 35, wherein the integration structure comprises an interference fitting.

46. The dermal delivery apparatus of claim 35, wherein the integration structure comprises a latch.
